(19) United States
(12) Patent Application Publication
(10) Pub. No.: US 2026/0251382 A1
(43) Pub. Date: Aug. 27, 2026

(54) BIOLOGICAL SAMPLE STORAGE DEVICE AND INLET-OUTLET HOUSING COMPONENT THEREOF

(71) Applicant: GENEPOINT TECHNOLOGIES (SHANGHAI) CO., LTD., Shanghai (CN)

(72) Inventors: Zhehua CHEN, Shanghai (CN); Fubin DING, Shanghai (CN); Haifeng YU, Shanghai (CN); Huang LI, Shanghai (CN)

(73) Assignee: GENEPOINT TECHNOLOGIES (SHANGHAI) CO., LTD., Shanghai (CN)

(21) Appl. No.: 19/648,098

(22) Filed: Apr. 15, 2026

Related U.S. Application Data
(63) Continuation of application No. PCT/CN2024/113590, filed on Aug. 21, 2024.

(30) Foreign Application Priority Data
Oct. 16, 2023 (CN) 202322772365.3
Oct. 16, 2023 (CN) 202322772514.6
Oct. 19, 2023 (CN) 202322815626.5
Oct. 20, 2023 (CN) 202322829849.7
Nov. 13, 2023 (CN) 202323059634.8
Mar. 28, 2024 (CN) 202420631023.1

(51) Int. Cl. F25D 25/04; F25D 17/06; F25D 21/14; F25D 23/02; F25D 23/06

(52) U.S. Cl. CPC F25D 25/04; F25D 17/06; F25D 21/14; F25D 23/026; F25D 23/028; F25D 23/066; F25D 2321/144; F25D 2331/804

(57) ABSTRACT

The present disclosure provides a biological sample storage device and an inlet-outlet housing component thereof. The biological sample storage device includes a box body defining an accommodation cavity for accommodating a biological sample. The accommodation cavity is provided with an inlet-outlet for entry and exit of the biological sample. A transfer mechanism is disposed at a position of the box body corresponding to the inlet-outlet. The transfer mechanism is configured to achieve transferring of the biological sample. The inlet-outlet housing component of the biological sample storage device includes a housing defining a housing cavity. When the housing is mounted on the box body, the housing cavity accommodates at least a portion of the transfer mechanism.

### CROSS-REFERENCES TO RELATED APPLICATIONS

This application is a Continuation of International Application No. PCT/CN2024/113590, filed on August 21, 2024, which claims priority to Chinese Application No. 202322772365.3, filed on October 16, 2023, Chinese Application No. 202322772514.6, filed on October 16 , 2023, Chinese Application No. 202322815626.5, filed on October 19, 2023, Chinese Application No. 202322829849.7, filed on October 20, 2023, Chinese Application No. 202323059634.8, filed on November 13, 2023, and Chinese Application No. 202420631023.1, filed on March 28, 2024, the entire contents of each of which are incorporated herein by reference.

### TECHNICAL FIELD

The present disclosure relates to the technical field of biological sample storage, and in particular to a biological sample storage device and an inlet-outlet housing component thereof.

### BACKGROUND

With the rapid development of the biomedical industry, large biobanks are emerging, and the storage of biological samples is increasing. Biological samples such as tissues and cells in clinical or laboratory settings are generally stored in a biological sample storage device. In the related technologies, in order to improve the automation of the storage process, the biological sample storage device may be provided with a transfer mechanism with a housing component to transfer the biological samples.

### SUMMARY

One or more embodiments of the present disclosure provide an inlet-outlet housing component of a biological sample storage device. The biological sample storage device may include a box body defining an accommodation cavity for accommodating a biological sample. The accommodation cavity may be provided with an inlet-outlet for entry and exit of the biological sample. A transfer mechanism may be disposed at a position of the box body corresponding to the inlet-outlet. The transfer mechanism may be configured to realize transferring of the biological sample. The inlet-outlet housing component may include a housing defining a housing cavity. When the housing is mounted on the box body, the housing cavity may accommodate at least a portion of the transfer mechanism.

In some embodiments, the housing cavity may be provided with a top opening. The transfer mechanism may be configured to transfer the biological sample between the top opening and the inlet-outlet.

In some embodiments, the top opening may be a stepped opening.

In some embodiments, the inlet-outlet housing component may further comprise a door body. The door body may be configured to open or close the top opening.

In some embodiments, the door body may include a door housing. A door housing cavity may be defined in the door housing. A heat preservation device may be disposed in the door housing cavity.

In some embodiments, a sealing strip may be disposed in a region of the door body corresponding to the top opening.

In some embodiments, the inlet-outlet housing component may further comprise an automatic opening mechanism fixed to the housing and located outside the housing cavity. The automatic opening mechanism may be configured to open or close the door body.

In some embodiments, plurality of rollers may be disposed at a bottom of the housing.

In some embodiments, the housing and the box body may be detachably connected through at least one of a threaded structure, an adhesive structure, or a snap-fit structure.

In some embodiments, the inlet-outlet housing component may further comprise an inner liner disposed in the housing cavity and fixedly connected with the housing, a gap being formed between the inner liner and the housing; and a heat preservation device disposed in the gap between the housing and the inner liner and configured to reduce cold energy of the housing cavity transferred to the outside of the housing cavity.

In some embodiments, the housing may be provided with a mounting mechanism. The mounting mechanism may be configured to be detachably connected with the box body. When the housing is mounted on the box body, the housing cavity may accommodate the transfer mechanism.

In some embodiments, the housing may include a body. The body may define the housing cavity. One side of the mounting mechanism may be detachably connected with the body, and the other side of the mounting mechanism may be detachably connected with the box body.

In some embodiments, at least one first threaded hole may be disposed on one side of the mounting mechanism, and the one side of the mounting mechanism may be detachably connected with the body through the at least one first threaded hole. At least one second threaded hole may be disposed on the other side of the mounting mechanism, and the other side of the mounting mechanism may be detachably connected with the box body through the at least one second threaded hole.

In some embodiments, an arrangement direction of a connecting line between any one of the at least one first threaded hole and any one of the at least one second threaded hole may not perpendicular to a length direction of the mounting mechanism.

In some embodiments, the mounting mechanism may include at least a first mounting portion and a second mounting portion which are located on two opposite sides of the body.

In some embodiments the mounting mechanism and the box body may be detachably connected through at least one of the threaded structure, the adhesive structure, or the snap-fit structure.

In some embodiments, the housing cavity may be provided with an opening. The transfer mechanism may be configured to achieve transferring of the biological sample between the opening and the inlet-outlet.

In some embodiments, the housing may be configured to be mounted on the box body. The housing may define the housing cavity with the opening. The housing cavity may be configured to accommodate the transfer mechanism. A cold energy generation device may be disposed in the housing cavity.

In some embodiments, the inlet-outlet housing component may further comprise a partition portion. The partition portion may be disposed in the housing cavity to separate a refrigeration cavity from the housing cavity. The cold energy generation device and a refrigeration fan may be disposed in the refrigeration cavity. The refrigeration cavity may be provided with an air inlet and an air outlet. The refrigeration fan may be configured to allow at least a portion of air to enter the refrigeration cavity through the air inlet and flow out of the air outlet after passing through the cold energy generation device.

In some embodiments, the air outlet may be located below the air inlet.

In some embodiments, the cold energy generation device may include an evaporator. A partition plate may be disposed in the housing cavity. The partition plate may be configured to separate the housing cavity into a first sub-cavity and a second sub-cavity. The partition portion may be disposed in the first sub-cavity. The second sub-cavity may be provided with a compressor and a condenser which are connected with the evaporator.

In some embodiments, the second sub-cavity may be further provided with a heat dissipation fan and a water receiving box for receiving defrost water of the refrigeration cavity. The water receiving box may be located at an air inlet end of the heat dissipation fan.

In some embodiments, the first sub-cavity may be located above the second sub-cavity. A gap may be formed between a lower end of the partition portion and the partition plate to form the air outlet.

In some embodiments, the housing may be detachably connected with the box body.

In some embodiments, an anti-condensation pipe may be disposed between the compressor and the condenser. The anti-condensation pipe may be disposed at a connection between the housing and the box body.

In some embodiments, the partition portion may be fixed on a sidewall of the housing. The partition portion may define the housing cavity with the sidewall of the housing.

In some embodiments, a horizontal side of the housing cavity may be provided with at least one of the air inlet or the air outlet.

In some embodiments, the opening may be located at a top of the housing.

In some embodiments, the housing may be configured to be mounted on the box body. The housing cavity may be provided with the opening. The partition plate may be disposed in the housing cavity. The partition plate may separate the housing cavity into the first sub-cavity and the second sub-cavity. The first sub- cavity may be configured to accommodate the transfer mechanism. The inlet-outlet housing component may further comprise a refrigeration system including an evaporator, a compressor, and a condenser which are connected with each other, the evaporator being disposed in the first sub-cavity, and at least one of the compressor or the condenser being disposed in the second sub-cavity; and the water receiving box disposed in the second sub-cavity and configured to receive defrost water of the first sub-cavity.

In some embodiments, the inlet-outlet housing component may further comprise the heat dissipation fan disposed in the second sub-cavity. The water receiving box may be located at an air outlet end of the heat dissipation fan.

In some embodiments, the condenser may be located at the air inlet end of the heat dissipation fan. The compressor may be located at the air outlet end of the heat dissipation fan.

In some embodiments, the water receiving box may be closer to the heat dissipation fan than the compressor.

In some embodiments, a drain port may be disposed at a bottom of the first sub-cavity. One end of the drain port may be connected with a drain pipe, and the other end of the drain pipe may be disposed above the water receiving box.

In some embodiments, the inlet-outlet housing component may further comprise the inner liner disposed in the housing cavity, a heat insulation material being disposed in at least a portion of a region between the inner liner and the housing.

In some embodiments, the housing may be detachably connected with the box body.

In some embodiments, the anti-condensation pipe may be disposed between the compressor and the condenser. The anti-condensation pipe may be disposed at the connection between the housing and the box body.

In some embodiments, the opening may be located at a top of the housing.

One or more embodiments of the present disclosure provide a biological sample storage device. The biological sample storage device may comprise a box body and an inlet-outlet housing component described any embodiment of the present disclosure. The box body may define an accommodation cavity for accommodating a biological sample. The accommodation cavity may be provided with an inlet-outlet for entry and exit of the biological sample. A transfer mechanism may be disposed at a position of the box body corresponding to the inlet-outlet. The transfer mechanism may be configured to achieve transferring of the biological sample.

In some embodiments, the housing cavity may be provided with a top opening. The transfer mechanism may be configured to transfer the biological sample between the top opening and the inlet-outlet.

In some embodiments, a mounting mechanism of the inlet-outlet housing component may be configured to be detachably connected with the box body. When a housing of the inlet-outlet housing component is mounted on the box body, a housing cavity of the inlet-outlet housing component may accommodate the transfer mechanism.

In some embodiments, the inlet-outlet housing component may include a housing. The housing may be mounted on the box body. The housing may define the housing cavity connected with the accommodation cavity through the inlet-outlet. The housing cavity may be provided with an opening for the biological sample to enter and exit the housing cavity. The inlet-outlet and the opening may be located at different positions in at least two directions. A height of the opening may be greater than a height of the inlet-outlet. The transfer mechanism may be disposed in the housing cavity and configured to drive the biological sample to transfer between the inlet-outlet and the opening in the at least two directions.

In some embodiments, a side of the accommodation cavity may be provided with the inlet-outlet. The housing may be mounted on a sidewall of the box body.

In some embodiments, the transfer mechanism may include a bearing portion. The bearing portion may be configured to bear the biological sample. The transfer mechanism may further include a first transmission portion configured to drive the bearing portion to move vertically and a second transmission portion configured to drive the bearing portion to move horizontally.

In some embodiments, the first transmission portion may include a first driving device. An output shaft of the first driving device may be connected with a first driving wheel. The first driving wheel may be connected with a first driven wheel through a first transmission belt. The first transmission belt may be connected with the second transmission portion through a connection member to drive the second transmission portion to move vertically.

In some embodiments, the connection member may be connected with the box body through a first guide rail.

In some embodiments, the connection member may be slidably connected with the bearing portion. The second transmission portion may include a second driving device. An output shaft of the second driving device may be connected with a second driving wheel. The second driving wheel may be connected with a second driven wheel through a second transmission belt. The second transmission belt may be connected with the bearing portion to drive the bearing portion to move horizontally.

In some embodiments, the connection member may be connected with the bearing portion through a second guide rail.

In some embodiments, the opening may be disposed at a top of the housing.

In some embodiments, the housing may be detachably connected with the box body.

In some embodiments, the inlet-outlet housing component may further include an inner liner disposed in the housing cavity and detachably connected with the housing. A heat insulation material may be disposed between the housing and the inner liner.

In some embodiments, the transfer mechanism may be configured to move the biological sample into and/or out of the accommodation cavity. The transfer mechanism may be configured to have a first state of moving from outside of the accommodation cavity to inside of the accommodation cavity, and a second state of moving from the inside of the accommodation cavity to the outside of the accommodation cavity. The biological sample storage device may further comprise a first door body. The first door body may be disposed at the inlet-outlet for opening and closing the inlet-outlet and configured to gradually open the inlet-outlet when the transfer mechanism is in the first state, and gradually close the inlet-outlet when the transfer mechanism is in the second state.

In some embodiments, the biological sample storage device may further comprise an elastic member connecting the first door body and the box body, such that the first door body may gradually close the inlet-outlet through an elasticity of the elastic member.

In some embodiments, the first door body may be arranged in an inclined manner such that the first door body may gradually close the inlet-outlet through gravity.

In some embodiments, at least one of the first door body or the transfer mechanism may be provided with a magnetic adsorption portion. The magnetic adsorption portion may be configured to make the first door body and the transfer mechanism adsorb to each other such that the first door body may gradually close the inlet-outlet through a magnetic force.

In some embodiments, the transfer mechanism may be provided with a fixing portion. The fixing portion may be configured to fix a biological sample cryopreservation box. The biological sample cryopreservation box may be configured to store the biological sample. The transfer mechanism may further include a moving portion. The moving portion may be connected with the fixing portion to drive the fixing portion to move into and/or out of the accommodation cavity. The fixing portion may gradually open the inlet-outlet by pressing the first door body.

In some embodiments, the biological sample storage device may further comprise at least one roller. Each of the at least one roller may be mounted on the fixing portion. The fixing portion may press against the first door body through the at least one roller.

In some embodiments, the first door body may include a door panel, a first end of the door panel being hinged to the box body; and a wedge block. A first end of the wedge block may be fixed to the door panel. A second end of the wedge block opposite to the first end of the wedge block may be configured to be pressed by the at least one roller. The second end of the wedge block may be an inclined surface. A distance between a portion of the second end of the wedge block that is close to the first end of the door panel and the door panel may be less than a distance between a portion of the second end of the wedge block that is away from the first end of the door panel and the door panel.

In some embodiments, the at least one roller may include a plurality of rollers. Arrangement directions of the plurality of rollers may be different from a movement direction of the moving portion.

In some embodiments, the biological sample storage device may further comprise a taking and placing device disposed in the accommodation cavity and configured to place the biological sample cryopreservation box stored in the accommodation cavity on the fixing portion, or take the biological sample cryopreservation box fixed to the fixing portion to be stored in the accommodation cavity.

In some embodiments, the biological sample storage device may further comprise a second door body disposed at the inlet-outlet and away from the accommodation cavity relative to the first door body.

### BRIEF DESCRIPTION OF THE DRAWINGS

The present disclosure will be further illustrated by way of exemplary embodiments, which will be described in detail by means of the accompanying drawings. In these embodiments, the same numbering indicates the same structure or the same operation.

FIG. 1 is a schematic structural diagram illustrating a biological sample storage device according to some embodiments of the present disclosure;

FIG. 2 is a schematic structural diagram illustrating an inlet-outlet housing component according to some embodiments of the present disclosure.

FIG. 3 is a schematic structural diagram illustrating an inlet-outlet housing component with a door body hidden according to some embodiments of the present disclosure;

FIG. 4 is a schematic diagram illustrating an inlet-outlet housing component provided with a transfer mechanism according to some embodiments of the present disclosure;

FIG. 5 is a schematic structural diagram illustrating a first mounting portion of an inlet-outlet housing component according to some embodiments of the present disclosure;

FIG. 6 is a schematic diagram illustrating an inlet-outlet housing component from a first viewing angle according to some embodiments of the present disclosure;

FIG. 7 is a schematic diagram illustrating an inlet-outlet housing component from a second viewing angle according to some embodiments of the present disclosure;

FIG. 8 is a schematic structural diagram illustrating the inlet-outlet housing component shown in FIG. 7 with a partition portion hidden ;

FIG. 9 is a cross-sectional diagram illustrating a biological sample storage device according to some embodiments of the present disclosure;

FIG. 10 is a cross-sectional diagram illustrating a biological sample storage device according to some embodiments of the present disclosure;

FIG. 11 is a cross-sectional diagram illustrating a biological sample storage device according to some embodiments of the present disclosure;

FIG. 12 is a schematic diagram illustrating a transfer mechanism of a biological sample storage device from a first viewing angle according to some embodiments of the present disclosure;

FIG. 13 is a schematic diagram illustrating a transfer mechanism of a biological sample storage device from a second viewing angle according to some embodiments of the present disclosure;

FIG. 14 is a schematic diagram illustrating a biological sample storage device with a portion of a box body hidden from a first viewing angle according to some embodiments of the present disclosure;

FIG. 15 is a schematic diagram illustrating a biological sample storage device with a portion of a box body hidden from a second viewing angle according to some embodiments of the present disclosure; and

FIG. 16 is a schematic diagram illustrating an enlargement of a region A shown in FIG. 15.

Reference signs in the figures: 1000 represents a biological sample storage device, 100 represents a box body, 110 represents an inlet-outlet, 120 represents a door body of an inlet-outlet, 121 represents a door panel, 122 represents a wedge block, 200 represents an inlet-outlet housing component, 210 represents a housing, 211 represents a housing cavity, 212 represents an opening, 213 represents a first threaded hole, 214 represents a second threaded hole, 215 represents a first mounting portion, 216 represents a second mounting portion, 217 represents a body, 218 represents a mounting mechanism, 219 represents a partition plate, 220 represents a roller, 230 represents a door body, 231 represents an automatic opening mechanism, 240 represents a partition portion, 241 represents a cold energy generation device, 242 is a refrigeration fan, 243 represents an air inlet, 244 represents an air outlet, 250 represents a compressor, 260 represents a condenser, 270 represents a heat dissipation fan, 280 represents a water receiving box, 300 represents a transfer mechanism, 310 represents a bearing portion, 321 represents a first driving device, 323 represents a first transmission belt, 326 represents a first guide rail, 331 represents a second driving device, 332 represents a second driving wheel, 333 represents a second transmission belt, 334 represents a second driven wheel, 335 represents a second guide rail, 340 represents a fixing portion, and 350 represents a roller of the fixing portion.

### DETAILED DESCRIPTION

In order to more clearly illustrate the technical solutions of the embodiments of the present disclosure, the embodiments are described in detail below with reference to the accompanying drawings. Obviously, the contents described below are some examples or embodiments of the present disclosure. It is possible for a person of ordinary skill in the art to apply the technical solutions or means disclosed in the present disclosure to other similar scenarios in accordance with these technical contents.

It should be understood that the terms “system”, “device”, “equipment”, “part” and/or “member” used herein are a way to distinguish between different components, elements, parts, sections, or assemblies at different levels. However, the terms may be replaced by other expressions if other words accomplish the same purpose.

Unless otherwise specified, technical terms used in the present disclosure to describe components, elements, etc., do not refer specifically to the singular, but may also include the plural. Generally, the terms “including” and “comprising” suggest only the inclusion of clearly identified steps and elements, however, the steps and elements do not constitute an exclusive list, and the method or apparatus may also include other steps or elements.

In the description of present disclosure, it should be understood that the description of orientation, such as the orientation or position relationship indicated by up, down, front, rear, left, right, etc., is based on the orientation or position relationship shown in the drawings, which is only for the convenience of describing this application and simplifying the description, and does not indicate or imply that the device or element referred to must have a specific orientation, be constructed and operated in a specific orientation, and therefore cannot be understood as a limitation on this application. In the description of the present disclosure, unless otherwise clearly defined, the terms such as setting, mounting, connecting, etc. should be understood in a broad sense, and a person skilled in the art can reasonably determine the specific meaning of the above terms in the present disclosure in combination with the specific content of the technical solution.

With the rapid development of the biomedical industry, large biobanks are emerging, and the storage of biological samples is increasing. Biological samples such as tissues and cells in clinical or laboratory settings are generally stored in a biological sample storage device for storing the biological samples. In the related art, in order to improve the automation of the storage process, the biological sample storage device may be provided with a transfer mechanism with a housing component to transfer the biological sample. However, such biological sample storage device is often not conducive to the preservation of the biological samples.

Some embodiments of the present disclosure provide an inlet-outlet housing component of a biological sample storage device. In some embodiments, the biological sample storage device may include a box body which defines an accommodation cavity for accommodating a biological sample. The housing cavity may be provided with an inlet-outlet for entry and exit of the biological sample. A transfer mechanism may be disposed at a position of the box body corresponding to the inlet-outlet. The transfer mechanism may be configured to achieve transferring of the biological sample. The inlet-outlet housing component may include a housing. The housing may define a housing cavity. When the housing is mounted on the box body, the housing cavity may accommodate at least a portion of the transfer mechanism.

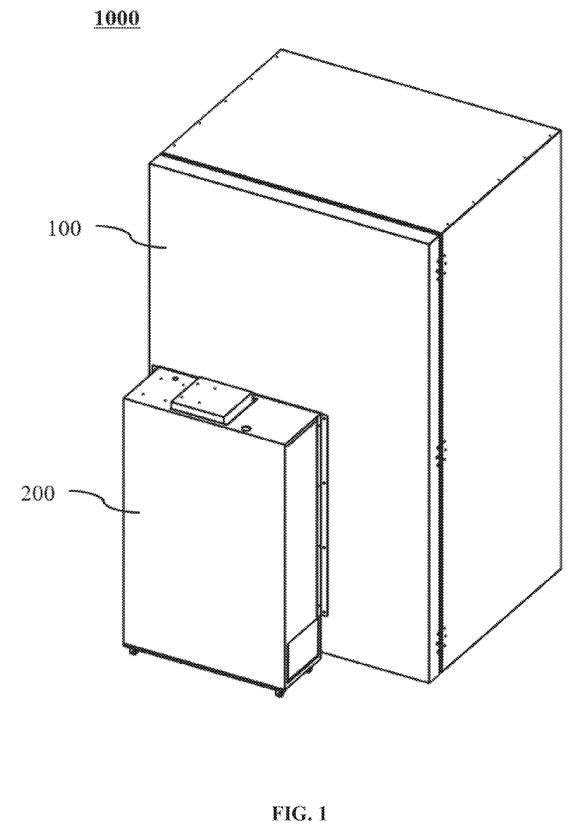

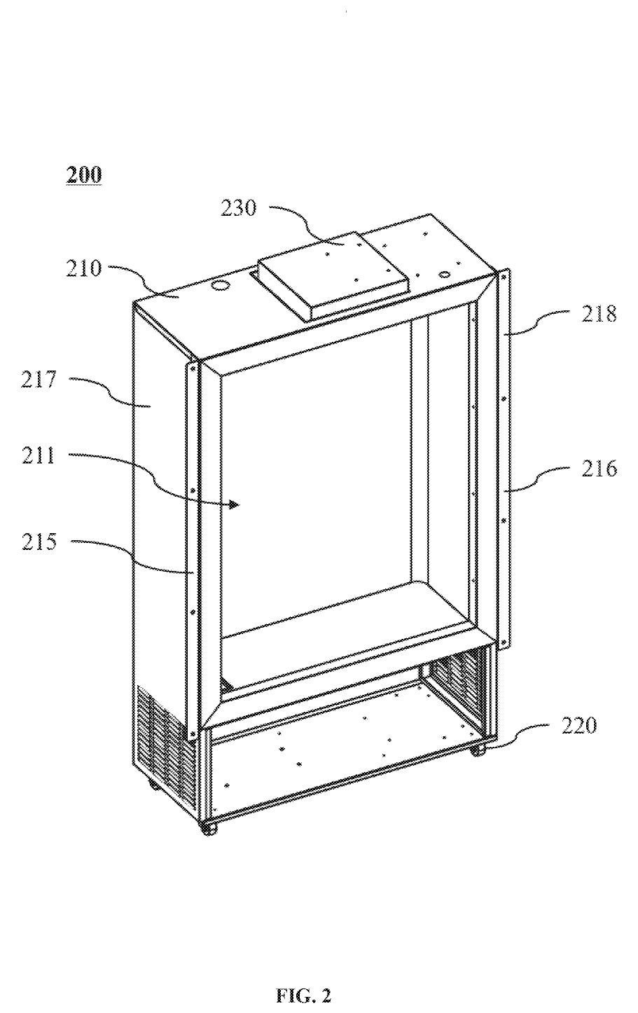

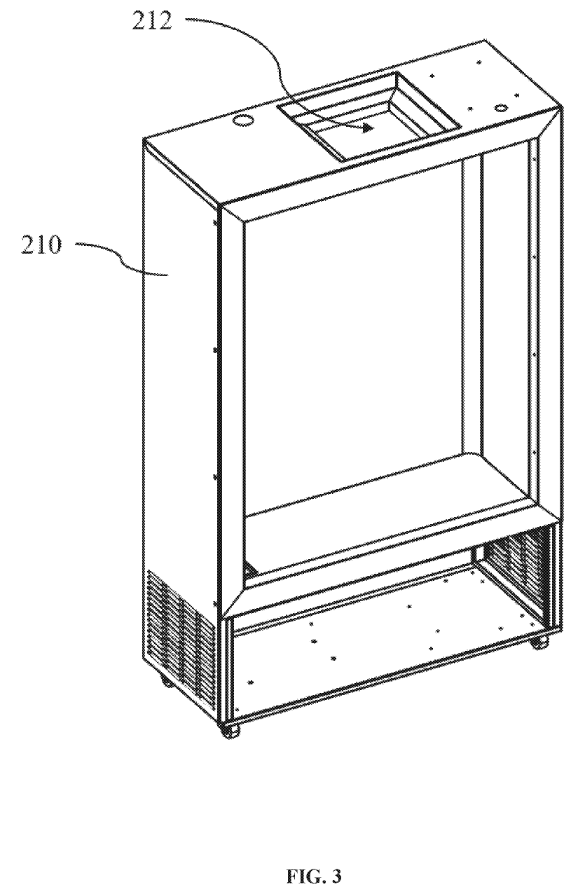

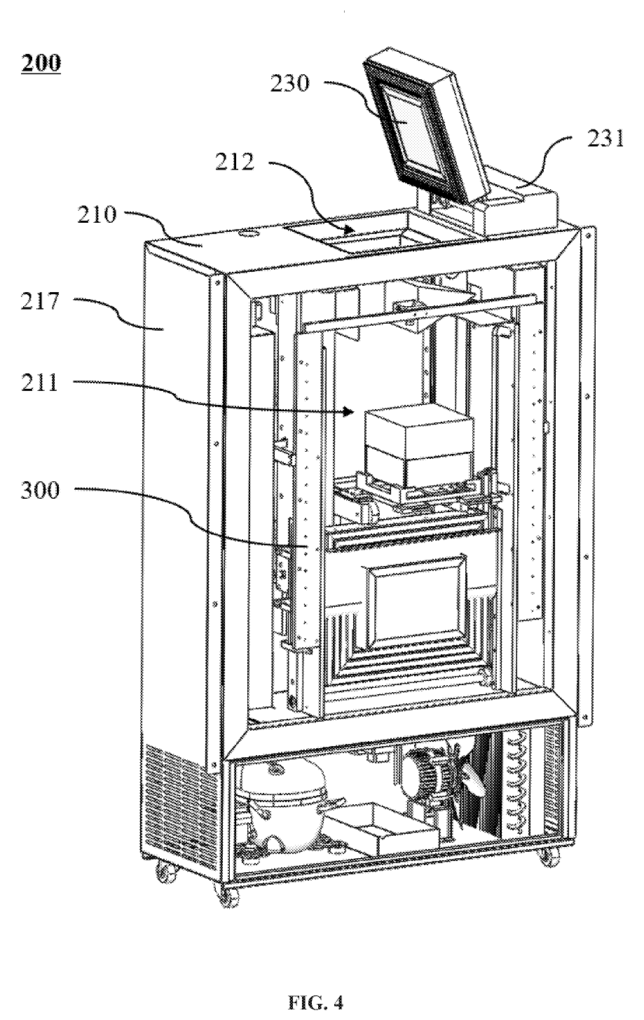

FIG. 1 is a schematic structural diagram illustrating a biological sample storage device according to some embodiments of the present disclosure. FIG. 2 is a schematic structural diagram illustrating an inlet-outlet housing component according to some embodiments of the present disclosure. FIG. 3 is a schematic structural diagram illustrating an inlet-outlet housing component with a door body hidden according to some embodiments of the present disclosure. FIG. 4 is a schematic diagram illustrating an inlet-outlet housing component provided with a transfer mechanism according to some embodiments of the present disclosure. In some embodiments, a biological sample storage device 1000 may be a refrigerator, a freezer, a cold storage, a liquid nitrogen storage system, etc. The biological sample storage device 1000 may also be referred to as a refrigeration and freezing device for storing a biological sample.

In some embodiments, the biological sample storage device 1000 may include a box body 100 which defines an accommodation cavity for accommodating the biological sample. The accommodation cavity may be provided with an inlet-outlet 110 for entry and exit of the biological sample. A transfer mechanism 300 may be disposed at a position of the box body 100 corresponding to the inlet-outlet. The transfer mechanism may be configured to achieve transferring of the biological sample. In some embodiments, an inlet-outlet housing component 200 may include a housing 210. The housing 210 may define a housing cavity 211. The housing cavity 211 may be provided with an opening 212. The opening 212 may be a top opening. The housing cavity 211 may accommodate at least a portion of the transfer mechanism 300. In some embodiments, the housing cavity 211 may accommodate the transfer mechanism, and the transfer mechanism may be configured to transfer the biological sample between the top opening and the inlet-outlet. In some embodiments, the housing 210 may be a cuboid structure. For example, the housing 210 may include side plate and a top plate connected with each other, and the opening 212 may be disposed on the top plate. In some embodiments, the housing 210 may be in other shapes. For example, the housing may be in a cylindrical shape, a table shape, a cone shape, etc. In some embodiments, when the housing 210 is in other shapes, the opening 212 may be disposed at a highest position of the housing 210.

According to the biological sample storage device 1000 and the inlet-outlet housing component 200 thereof provided in some embodiments of the present disclosure, the housing cavity 211 may be provided with the top opening. Since cold air naturally sinks, the top opening reduces outward diffusion of cold air inside the inlet-outlet housing component 200, and reduces inward diffusion of external hot air to the inside, thereby facilitating the storage of the biological sample. In addition, the humidity of the cold air inside the inlet-outlet housing component 200 is low, and the humidity of the hot air outside the inlet-outlet housing component 200 is high. The top opening also reduces internal frost. Frost affects the operation of one or more internal mechanisms (e.g., the transfer mechanism) of the inlet-outlet housing component 200. The top opening can reduce the internal frost, thereby ensuring the smooth operation of the one or more internal mechanisms.

In some embodiments, the top opening may be a stepped opening. In this way, the cold air inside the inlet-outlet housing component 200 needs a more complex path to escape, thereby avoiding cold energy loss and improving the storage effect of biological sample. In some embodiments, the stepped opening may be a primary stepped hole, a secondary stepped hole, a tertiary stepped hole, etc. In some embodiments, as shown in FIG. 3, the stepped opening may be the primary stepped hole, which may include a primary step. In some embodiments, the stepped opening may be in a square hole shape to match a shape of a cryopreservation box.

In some embodiments, the inlet-outlet housing component 200 may further include a door body 230. The door body 230 may be configured to open or close the top opening. The door body 230 prevents cold energy from flowing out of the top opening, thereby improving the storage effect of the biological sample. In some embodiments, the door body 230 may include a door housing which defines a door housing cavity. A heat preservation device may be disposed in the door housing cavity. In this way, the door housing can prevent leakage of the cold energy in the inlet-outlet housing component 20, thereby improving the storage effect of the biological sample. In some embodiments, the door body 230 may match a shape of the opening 212. For example, the opening 212 may be in a square hole shape, and the door body 230 may be in a cuboid shape that matches the opening 212. In some embodiments, the opening 212 may be a stepped opening, and the door body 230 may be in a stepped shape that matches the stepped opening, thereby further preventing leakage of the cold energy in the inlet-outlet housing component 200.

In some embodiments, a sealing strip may be disposed in a region of the door body 230 corresponding to the top opening, which improves the airtightness of the door body 230 when the door body 230 is closed, and ensures the storage effect of the biological sample. In some embodiments, the sealing strip may be disposed at a bottom of the door body 230. When the door body 230 is closed at the top opening, the sealing strip may fit with a step portion of the top opening. In some embodiments, the sealing strip may be disposed at a side of the door body 230. When the door body 230 is closed at the top opening, the sealing strip may fit with a sidewall of the top opening. In some embodiments, the bottom and the side of the door body 230 may be provided with the sealing strip, respectively. In some embodiments, when the door body 230 is in a stepped shape that matches the stepped opening, the sealing strip may be disposed at the bottom of the door body 230 and/or on the step portion of the door body 230.

In some embodiments, the inlet-outlet housing component 200 may further include an automatic opening mechanism. The automatic opening mechanism may be fixed to the housing 210 and located outside the housing cavity 211, and the automatic opening mechanism may be configured to open or close the door body 230. In some embodiments, as shown in FIG. 4, the inlet-outlet housing component 200 may include an automatic opening mechanism 231. The automatic opening mechanism 231 may be fixed to the housing 210 and located outside the housing cavity 211. In some embodiments, the door body 230 may be rotatably connected to the automatic opening mechanism 231, and the automatic opening mechanism 231 may drive the door body 230 to rotate to open or close the door body 230.

In some embodiments, the automatic opening mechanism may be configured to drive the door body 230 to translate in a first direction away from or close to the opening 212 to open or close the opening 212. The door body 230 is translated, and even if the opening 212 is disposed at the top of the housing 210, there is no need to worry about the problem of knocking over the biological sample when a user accidentally places the biological sample on the door body 230 and opens the door. In some embodiments, the automatic opening mechanism may be further configured to first drive the door body 230 to translate in the first direction when opening the door body 230 and then drive the door body 230 to translate in a second direction perpendicular to the first direction, and first drive the door body 230 to translate in the second direction when closing the door body 230 and then drive the door body 230 to translate in the first direction, thereby facilitating operation for personnel when opening the door body 230. The first direction may be a vertical direction, and the second direction may be a horizontal direction. In some embodiments, the first direction and the second direction may be other directions. For example, the first direction may be other directions that are 45 degrees, 30 degrees, or 60 degrees from a horizontal plane.

In some embodiments, a plurality of rollers 220 may be disposed at a bottom of the housing 210 to facilitating placement and movement of the inlet-outlet housing component 200 after the inlet-outlet housing component 200 is removed. In some embodiments, when components (e.g., the transfer mechanism, a cold energy generation device, etc.) of the inlet-outlet housing component 200 need to be repaired, the inlet-outlet housing component 200 needs to be removed relative to the box for repair, and the rollers 220 facilitate removal, movement, repair, and remounting of the inlet-outlet housing component 200. In some embodiments, when the inlet-outlet housing component 200 is mounted on the box body 100, the rollers 220 may not contact the ground, and the inlet-outlet housing component 200 may be attached to the box body 100 to reduce an occupied space. In some embodiments, when the inlet-outlet housing component 200 is mounted on the box body 100, the rollers 220 may keep in contact with the ground, and the rollers may bear at least a portion of the gravity of the inlet-outlet housing component 200, thereby reducing load bearing of the box body 100.

In some embodiments, the housing 210 may be provided with a mounting mechanism 218. The mounting mechanism 218 may be detachably connected with the box body 100 through at least one of a threaded structure, an adhesive structure, or a snap-fit structure. The connection through the threaded structure is stable and firm, and can avoid accidental opening. The connection through the adhesive structure is easy to produce. The connection through the snap-fit structure is simple and convenient to use.

In some embodiments, the inlet-outlet housing component 200 may further include an inner liner disposed in the housing cavity 211 and fixedly connected with the housing 210, a gap being formed between the inner liner and the housing 210; and a heat preservation device disposed in the gap between the housing 210 and the inner liner and configured to reduce the cold energy of the housing cavity transferred to the outside of the housing cavity 211. By providing the inner liner and the heat preservation device, the cold energy in the inlet-outlet housing component 200 can be prevented from being diffused outward.

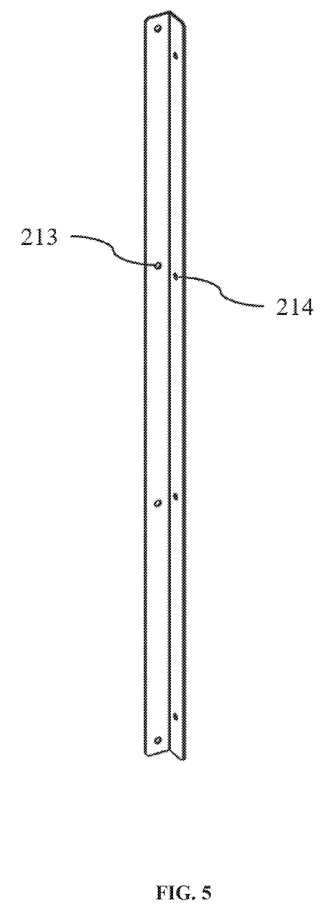

In some embodiments, in order to improve the automation of the storage process, a biological sample storage device may be provided with a transfer mechanism with a housing component to transfer a biological sample. However, such biological sample storage device is often not convenient for maintenance and transportation. Some embodiments of the present disclosure provide the inlet-outlet (or referred to as an inlet) housing component 200 of the biological sample storage device 1000 (or referred to as a refrigeration and freezing device). FIG. 5 is a schematic structural diagram illustrating a first mounting portion of an inlet-outlet housing component according to some embodiments of the present disclosure. In some embodiments, as shown in FIG. 1, FIG. 2, and FIG. 5, the inlet-outlet housing component 200 may include the housing 210. The housing 210 may define a housing cavity. The housing 210 may be provided with the mounting mechanism 218. The mounting mechanism 218 may be configured to be detachably connected with the box body 100. When the housing 210 is mounted on the box body 100, the housing cavity may accommodate the transfer mechanism.

According to the biological sample storage device 1000 and the inlet-outlet housing component 200 thereof provided in some embodiments of the present disclosure, the transfer mechanism is disposed on the box body 100, and the inlet-outlet housing component 200 is detachably connected relative to the box body 100, such that the transfer mechanism and other components can be inspected, repaired, replaced, etc. by disassembling the inlet-outlet housing component 200. In some embodiments, the transfer mechanism and the inlet-outlet housing component 200 are independent, i.e., the transfer mechanism is not fixed to the inlet-outlet housing component 200. Accordingly, it is easy to determine the use effect of the transfer mechanism after inspection, repair, replacement, etc. of the transfer mechanism and other components, thereby improving the user experience. In some embodiments, such detachable manner is also convenient for transportation of the biological sample storage device 1000. The biological sample storage device 1000 can be transported in a disassembled manner and then assembled after being transported to a desired place. In addition, the inlet-outlet housing component 200 can also avoid the diffusion of the cold energy and ensure the activity of the biological sample. In some embodiments, the transfer mechanism may be disposed in the inlet-outlet housing component 200.

In some embodiments, the housing 210 may include a body 217. The body 217 may define a housing cavity. One side of the mounting mechanism 218 may be detachably connected with the body 217, and the other side of the mounting mechanism 218 may be detachably connected with the box body 100, thereby facilitating production of the housing 210.

In some embodiments, at least one first threaded hole 213 may be disposed on one side of the mounting mechanism 218. The one side of the mounting mechanism 218 may be detachably connected with the body 217 through the at least one first threaded hole 213. At least one second threaded hole 214 may be disposed on the other side of the mounting mechanism 218. The other side of the mounting mechanism 218 may be detachably connected with the box body 100 through the at least one second threaded hole 214. The threaded connection is stable and firm, and can avoid accidental opening.

In some embodiments, an arrangement direction of a connecting line between any one of the at least one first threaded hole 213 and any one of the at least one second threaded hole 214 may not be perpendicular to a length direction of the mounting mechanism 218. For example, if the mounting mechanism 218 is in a strip shape, the arrangement direction of the connecting line between any one of the at least one first threaded hole 213 and any one of the at least one second threaded hole 214 may not be perpendicular to the length direction of the mounting mechanism 218. By staggering the at least one first threaded hole 213 and the at least one second threaded hole 214, the assembly of bolts is facilitated, and mutual interference of the bolts during the assembly is avoided, and thus a width of the mounting mechanism 218 does not need to be particularly large.

In some embodiments, the mounting mechanism 218 may include at least a first mounting portion 215 and a second mounting portion 216 located on two opposite sides of the body 217, respectively. For example, the first mounting portion 215 and the second mounting portion 216 may be mounted on left and right sides or upper and lower sides of the body 217, so as to ensure firm mounting. In some embodiments, the mounting mechanism 218 may be detachably connected with the box body 100 through at least one of a threaded structure, an adhesive structure, or a snap-fit structure. The connection through the threaded structure is stable and firm, and can avoid accidental opening. The connection through the adhesive structure is easy to produce. The connection through the snap-fit structure is simple and convenient to use.

In some embodiments, the housing cavity may be provided with a housing cavity opening. The transfer mechanism may be configured to achieve transferring of the biological sample between the housing cavity opening and the inlet-outlet, thereby improving the automation and enhancing the user experience.

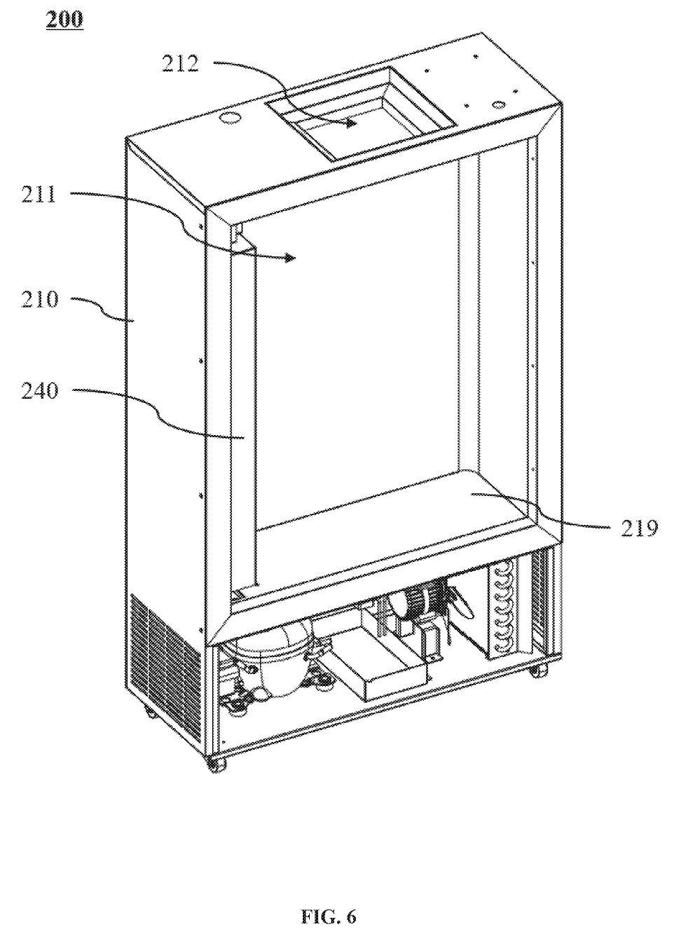

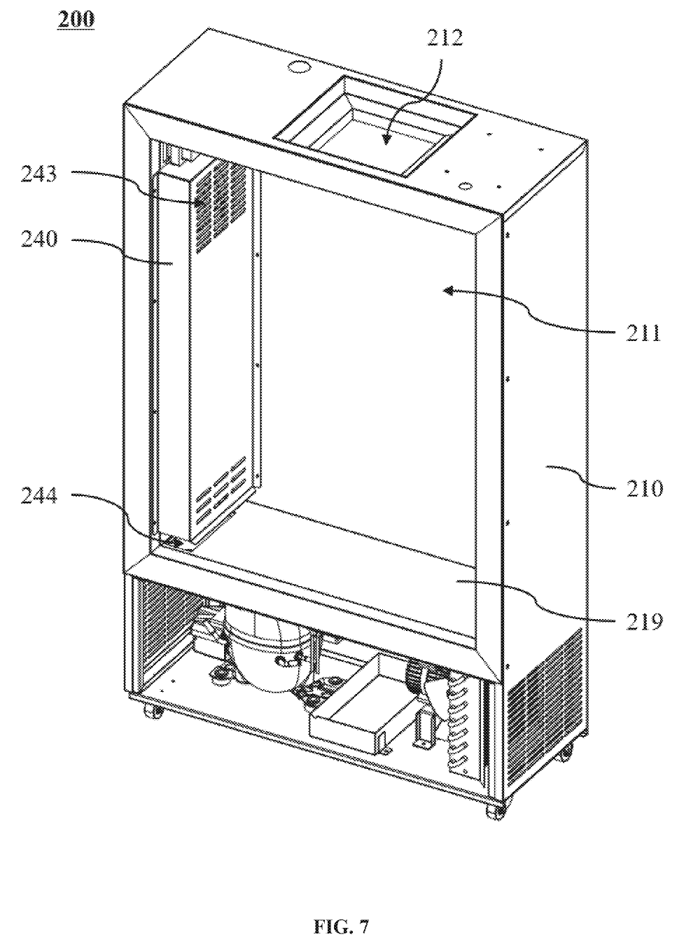

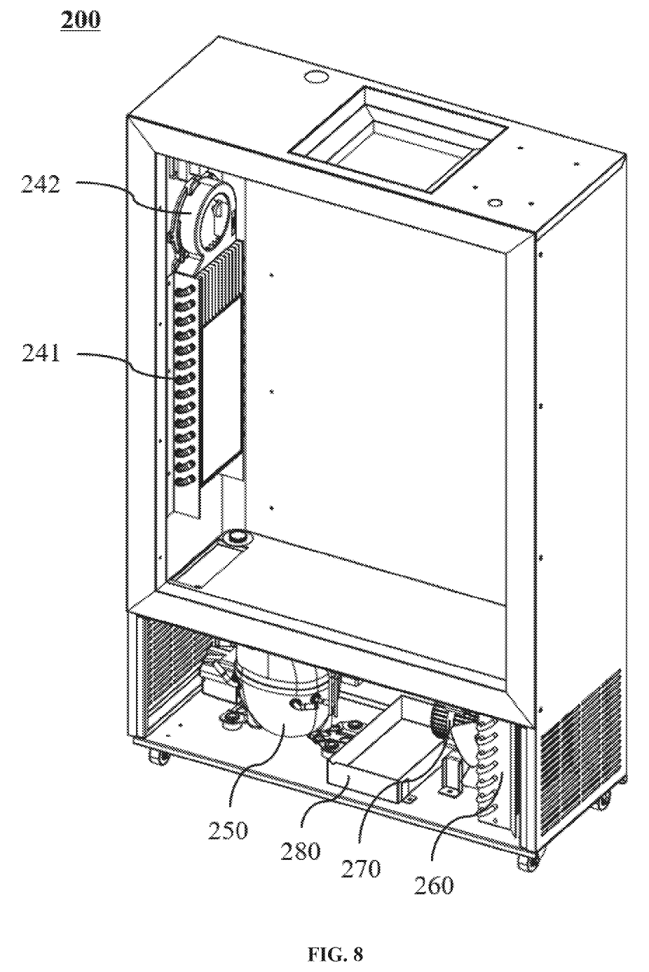

The embodiments of the present disclosure provide the inlet-outlet housing component 200 of the biological sample storage device (which may be a refrigerator, a cold storage, a freezer, a storage device with a liquid nitrogen tank, etc.). FIG. 6 is a schematic diagram illustrating an inlet-outlet housing component from a first viewing angle according to some embodiments of the present disclosure. FIG. 7 is a schematic diagram illustrating an inlet-outlet housing component from a second viewing angle according to some embodiments of the present disclosure. FIG. 8 is a schematic structural diagram illustrating the inlet-outlet housing component shown in FIG. 7 with a partition portion hidden. In some embodiments, the biological sample storage device may include a box body that defines an accommodation cavity for accommodating a biological sample (e.g., the biological sample may be accommodated in the form of a cryopreservation tube or a cryopreservation box). The accommodation cavity may be provided with an inlet-outlet for entry and exit of the biological sample. A transfer mechanism may be disposed at a position of the box body corresponding to the inlet-outlet. The transfer mechanism may be configured to achieve transferring of the biological sample.

In some embodiments, the inlet-outlet housing component 200 may include the housing 210. The housing 210 may be configured to be mounted on the box body. The housing 210 may define the housing cavity 211 provided with the opening 212. The housing cavity 211 may be configured to accommodate the transfer mechanism. In some embodiments, a cold energy generation device 241 may be disposed in the housing cavity 211. By providing the cold energy generation device 241 in the housing cavity 211, the air in the housing cavity 211 can be cooled. In some embodiments, the cold air can frost on the cold energy generation device, thereby dehumidifying the housing cavity 211.

In some embodiments, the inlet-outlet housing component 200 may include a partition portion 240 disposed in the housing cavity 211 to separate a refrigeration cavity from the housing cavity 211. The cold energy generation device 241 and a refrigeration fan 242 may be disposed in the refrigeration cavity. The refrigeration cavity may be provided with an air inlet 243 and an air outlet 244. The refrigeration fan 242 may be configured to allow at least a portion of air to enter the refrigeration cavity through the air inlet 243 and flow out of the air outlet 244 after passing through the cold energy generation device 241. In some embodiments, the air outlet 244 may be located below the air inlet 243. In some embodiments, the air outlet 244 may be located at a bottom of the refrigeration cavity.

The refrigeration cavity of the inlet-outlet housing component 200 is provided with the air inlet 243 and the air outlet 244 located below the air inlet 243. Due to the principle that hot air naturally rises and cold air naturally sinks, the biological sample storage device and the inlet-outlet housing component 200 thereof provided by the present disclosure can preferentially cool the air of a relatively high temperature, thereby improving the refrigeration efficiency, facilitating the reduction of a temperature inside the housing cavity 211, and thus facilitating the storage of the biological sample.

In some embodiments, the cold energy generation device 241 may include an evaporator. A partition plate 219 may be disposed in the housing cavity 211. The partition plate 219 may separate the housing cavity 211 into a first sub-cavity and a second sub-cavity. The partition portion 240 may be disposed in the first sub-cavity. The second sub-cavity may be provided with a compressor 250 and a condenser 260 which are connected with the evaporator. Such refrigeration manner has a high refrigeration efficiency and a wide refrigeration range.

In some embodiments, the cold energy generation device 241 may also be a portion that generates cold energy in a semiconductor refrigeration system, or a container for storing liquid nitrogen, etc.

In some embodiments, the second sub-cavity may be further provided with a heat dissipation fan 270 and a water receiving box 280 configured to receive defrost water of the refrigeration cavity. The water receiving box 280 may be located at an air inlet end of the heat dissipation fan 270. It can be understood that the heat dissipation fan 270 can take away heat of the compressor 250 and the condenser 260. The water receiving box 280 is disposed at the air inlet end of the cooling fan 270, such that the evaporation of water is facilitated, and water vapor is also taken away. By arranging the water receiving box 280 at the air inlet end of the heat dissipation fan 270, the heat dissipation efficiency of the condenser 260 can also be guaranteed.

In some embodiments, the first sub-cavity may be located above the second sub-cavity, and a gap may be disposed between a lower end of the partition portion 240 and the partition plate 219 to form the air outlet 244, thereby increasing the air volume. In some embodiments, a plurality of small holes may be disposed at the bottom of the partition portion 240 to assist in air outlet.

In some embodiments, the housing 210 may be detachably connected with the box body, thereby facilitating mounting and maintenance of the biological sample storage device. In some embodiments, an anti-condensation pipe may be disposed between the compressor 250 and the condenser 260. The anti-condensation pipe may be arranged at a connection between the housing 210 and the box body. In some embodiments, a portion of the condenser 260 may be taken as the anti-condensation pipe, and a size of the anti-condensation pipe may be set according to actual conditions. In some embodiments, the partition portion 240 may be fixed on a sidewall of the housing 210, and the partition portion 240 may define the housing cavity 211 with the sidewall of the housing 210. At least one of the air inlet 243 or the air outlet 244 may be disposed on a horizontal side of the housing cavity 211. The opening 212 may be located at the top of the housing 210, thereby preventing hot air with a high humidity from entering the housing cavity 211, and facilitating the storage of the biological sample.

In some embodiments, the inlet-outlet housing component 200 may include the housing 210, a refrigeration system, and the water receiving box 280. The housing 210 may be configured to be mounted on the box body. The housing 210 may define the housing cavity 211 provided with the opening 212. The partition plate 219 may be disposed in the housing cavity 211. The partition plate 219 may separate the housing cavity 211 into the first sub-cavity and the second sub-cavity. The first sub- cavity may be configured to accommodate the transfer mechanism. The refrigeration system may include the evaporator, the compressor 250, and the condenser 260 which are connected with each other. The evaporator may be disposed in the first sub-cavity. At least one of the compressor 250 or the condenser 260 may be disposed in the second sub-cavity. The water receiving box 280 may be disposed in the second sub-cavity and configured to receive the defrost water of the first sub-cavity.

The inlet-outlet housing component 200 can provide the cold energy to the housing cavity 211 to reduce the risk of the biological sample being transferred in the housing cavity 211, thereby facilitating the preservation of the biological sample. In addition, the defrost water of the evaporator of the first sub-cavity received in the water receiving box 280 can be evaporated into the air at a high temperature of the second sub-cavity without manually pouring out the defrost water, thereby improving the user experience.

In some embodiments, the inlet-outlet housing component 200 may further include the heat dissipation fan 270. The heat dissipation fan 270 may be disposed in the second sub-cavity. The water receiving box 280 may be located at the air outlet end of the heat dissipation fan 270. The heat dissipation fan 270 can improve the efficiency of heat dissipation, and arranging the water receiving box 280 at the air outlet end of the heat dissipation fan 270 is conducive to the evaporation of the defrost water.

In some embodiments, the condenser 260 may be located at the air inlet end of the heat dissipation fan 270, and the compressor 250 may be located at the air outlet end of the heat dissipation fan 270. In this way, the air inhaled by the heat dissipation fan 270 can perform better heat dissipation for the condenser 260, and also realize heat dissipation for the compressor 250, thereby maximizing the utilization rate of the air.

In some embodiments, the water receiving box 280 may be closer to the heat dissipation fan than the compressor 250, so as to better evaporate the defrost water.

In some embodiments, a drain port may be disposed at the bottom of the first sub-cavity. One end of the drain port may be connected with a drain pipe, and the other end of the drain pipe may be disposed at above the water receiving box 280, so as to guide a flow direction of the defrost water. Specifically, the drain port may be disposed at a position corresponding to the evaporator.

In some embodiments, the inlet-outlet housing component 200 may further include an inner liner disposed in the housing cavity, and a heat insulation material may be disposed in at least a portion of a region between the inner liner and the housing 210, thereby improving the heat insulation effect of the accommodation cavity and facilitating the storage of the biological sample.

Some embodiments of the present disclosure further provide the biological sample storage device 1000. The biological sample storage device 1000 may include the box body 100 and the inlet-outlet housing component 200 of any embodiment of the present disclosure. The box body 100 defines an accommodation cavity for accommodating a biological sample. The accommodation cavity may be provided with an inlet-outlet for entry and exit of the biological sample. A transfer mechanism may be disposed at a position of the box body 100 corresponding to the inlet-outlet. The transfer mechanism may be configured to achieve transferring of the biological sample. The inlet-outlet housing component 200 of any embodiment of the present disclosure may include the housing 210. The housing 210 may define the housing cavity 211. The housing cavity 211 may be provided with the opening 212. The opening 212 may be a top opening. The housing cavity 211 may accommodate the transfer mechanism. The transfer mechanism may be configured to transfer the biological sample between the top opening and the inlet-outlet. In some embodiments, the housing 210 of the inlet-outlet housing component 200 may be mounted on the box body. In some embodiments, the housing 210 may be provided with the mounting mechanism 218. The mounting mechanism 218 may be configured to be detachably connected with the box body 100. When the housing 210 is mounted on the box body 100, the housing cavity may accommodate the transfer mechanism.

In some embodiments, a refrigeration cavity of the biological sample storage device may be provided with the air inlet 243 and the air outlet 244 located below the air inlet 243. Due to the principle that hot air naturally rises and cold air naturally sinks, the biological sample storage device and the inlet-outlet housing component 200 thereof provided by the present disclosure can preferentially cool the air of a relatively high temperature, thereby improving the refrigeration efficiency, facilitating the reduction of a temperature inside the housing cavity 211, and thus facilitating the storage of the biological sample.

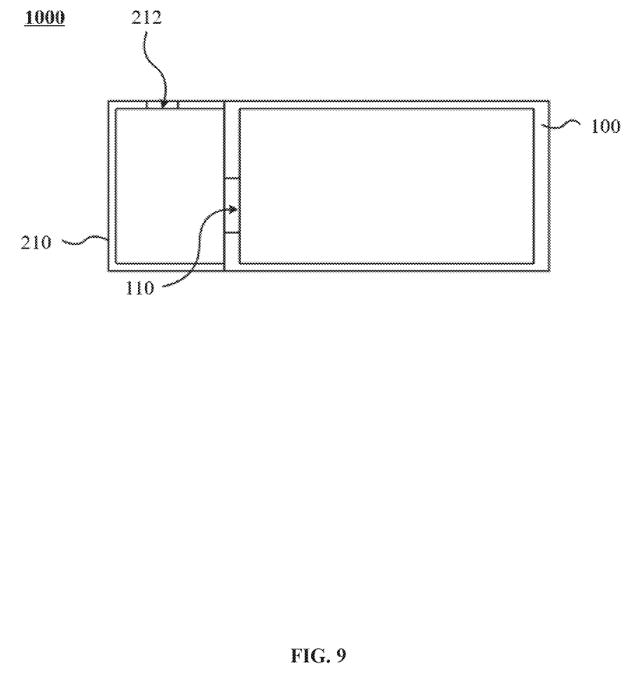

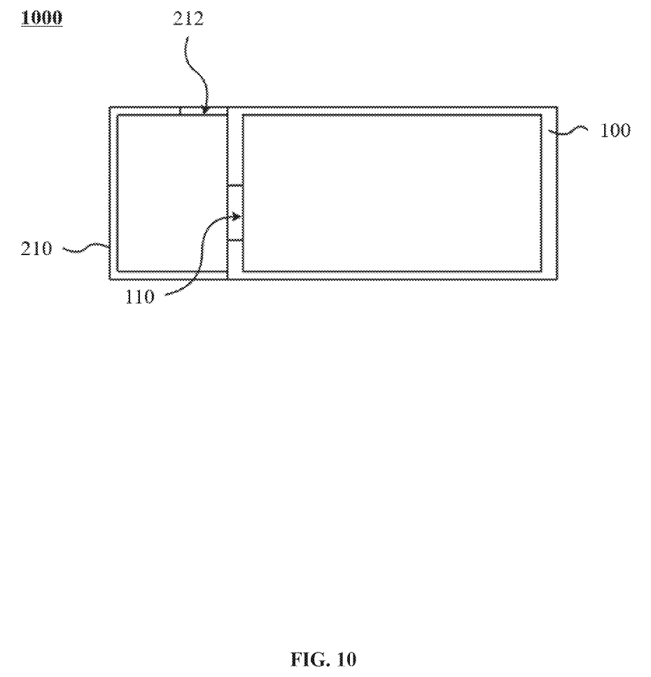

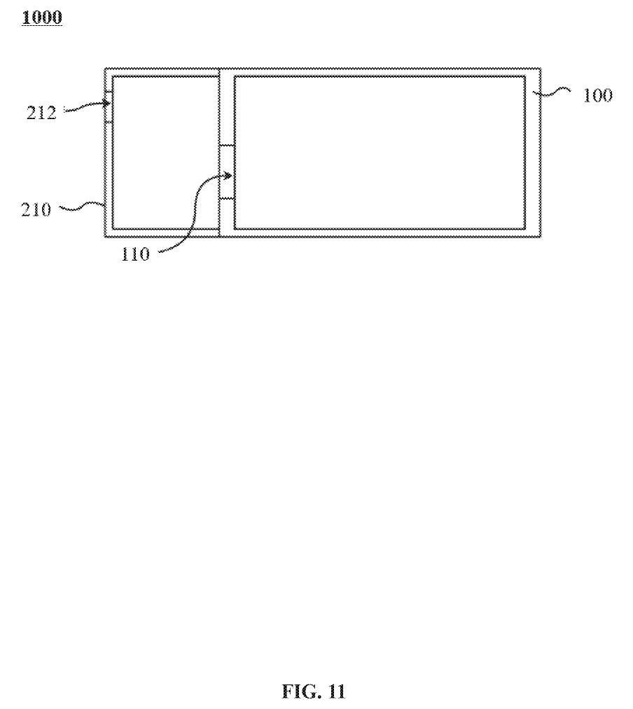

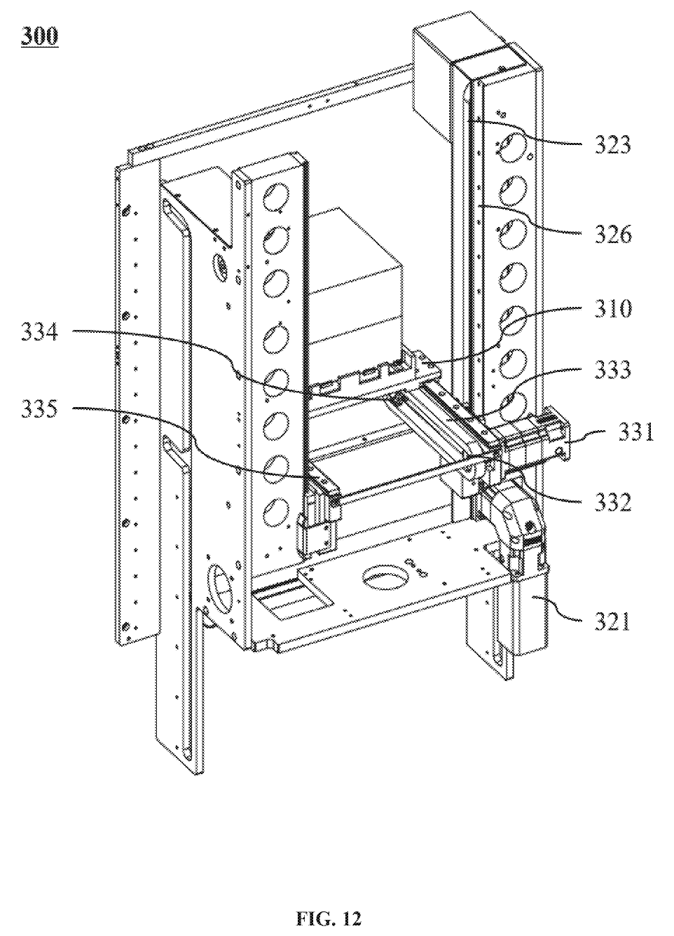

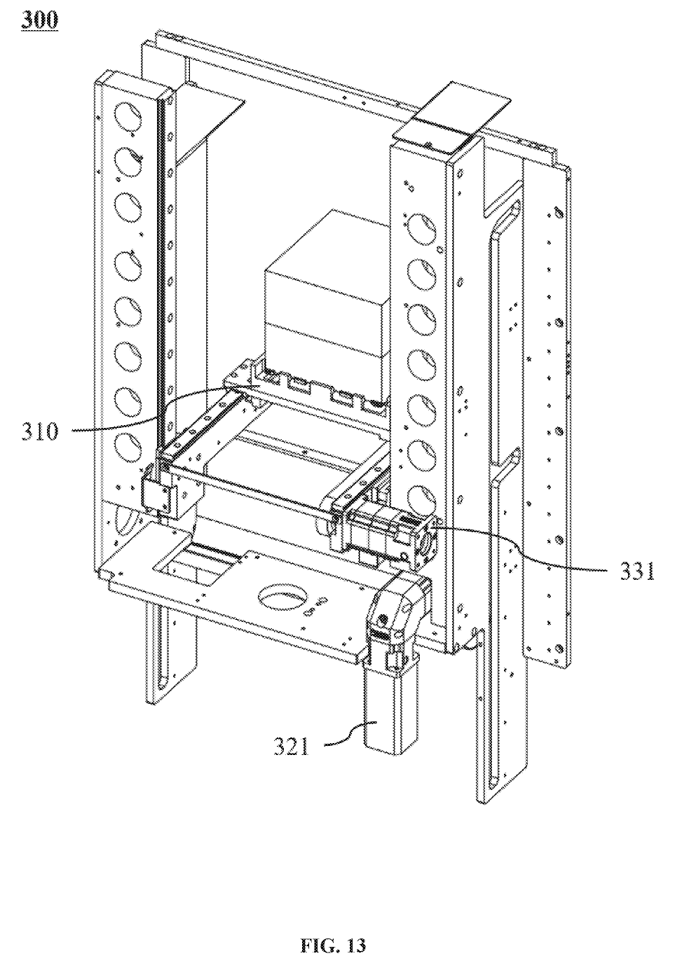

In some embodiments, the biological sample storage device is prone to leakage of the cold energy when taking and placing the biological sample, resulting in poor storage effect of the biological sample. Some embodiments of the present disclosure provide the biological sample storage device 1000. FIG. 9 is a cross-sectional diagram illustrating a biological sample storage device according to some embodiments of the present disclosure. FIG. 10 is a cross-sectional diagram illustrating a biological sample storage device according to some embodiments of the present disclosure. FIG. 11 is a cross-sectional diagram illustrating a biological sample storage device according to some embodiments of the present disclosure. FIG. 12 is a schematic diagram illustrating a transfer mechanism of a biological sample storage device from a first viewing angle according to some embodiments of the present disclosure. FIG. 13 is a schematic diagram illustrating a transfer mechanism of a biological sample storage device from a second viewing angle according to some embodiments of the present disclosure.

In some embodiments, as shown in FIGS. 9-13, the biological sample storage device 1000 may include the box body 100, the housing 210, and the transfer mechanism 300. The box body 100 may define an accommodation cavity for storing a biological sample. The accommodation cavity may be provided with the inlet-outlet 110 for entry and exit of the biological sample. The housing 210 may be mounted on the box body 100. The housing 210 may define a housing cavity connected with the accommodation cavity through the inlet-outlet 110. The housing cavity may be provided with the opening 212 for the biological samples to enter and exit the housing cavity. The inlet-outlet 110 and the opening 212 may be located at different positions in at least two directions. A height of the opening 212 may be greater than a height of the inlet-outlet 110. The transfer mechanism 300 may be disposed in the housing cavity and configured to drive the biological sample to transfer between the inlet-outlet 110 and the opening 212 in the at least two directions. In some embodiments, since a temperature of the accommodation cavity is lower than a temperature of the housing cavity, by setting the height of the opening 212 to be greater than the height of the inlet-outlet 110, the air with a relatively high temperature in the housing cavity entering the accommodation cavity can be reduced, thereby avoiding affecting the temperature in the accommodation cavity.

Since the positions of the inlet-outlet 110 and the opening 212 of the biological sample storage device 1000 are different in the at least two directions, compared with a situation that two inlet-outlets are located in different positions only in one direction, it is difficult for cold air to flow from the opening 212 to the inlet-outlet 110 and then flow out, thereby avoiding the leakage of the cold air and improving the storage effect of the biological sample. In addition, since hot air naturally rises and cold air naturally sinks, the positional relationship between the inlet-outlet 110 and the opening 212 can prevent the external hot air from entering the housing cavity, which further improves the storage effect of the biological sample. Since the humidity of the external hot air is relatively high, the positional relationship between the inlet-outlet 110 and the opening 212 can reduce frost inside the biological sample storage device 1000.

It can be understood that the at least two directions may refer to at least two of an up-down direction, a front-back direction, and a left-right direction of the biological sample storage device 1000. For example, the inlet-outlet 110 and the opening 212 may be located at different positions in the up-down direction and the front-back direction (as shown in FIG. 9, FIG. 10, and FIG. 11). That is, one of the inlet-outlet 110 and the opening 212 is located at an upper portion and the other of the inlet-outlet 110 and the opening 212 is located at a lower portion, and one of the inlet-outlet 110 and the opening 212 is located at a front and the other of the inlet-outlet 110 and the opening 212 is located at a back. It can be understood that the opening 212 may be located at any position of the housing 210, such as a top wall, a front wall, a sidewall, etc. As shown in FIG. 10, a rear end of the opening 212 may coincide with a front end of the inlet-outlet 110, but an overall position of the opening 212 may be in front of an overall position of the inlet-outlet 110, which means that the position of the opening 212 and the position of the inlet-outlet 110 may be different in the front-back direction of the biological sample storage device 1000. That is, the inlet-outlet 110 and the opening 212 may be located at different positions in the up-down direction and the front-back direction of the biological sample storage device 1000 (i.e., one of the inlet-outlet 110 and the opening 212 is located at the upper portion and the other of the inlet-outlet 110 and the opening 212 is located at the lower portion, and one of the inlet-outlet 110 and the opening 212 is located at left and the other of the inlet-outlet 110 and the opening 212 is located at right), or the inlet-outlet 110 and the opening 212 may be located at different positions in the up-down direction, the front-back direction, and the left-right direction of the biological sample storage device 1000 (i.e., one of the inlet-outlet 110 and the opening 212 is located at the upper portion and the other of the inlet-outlet 110 and the opening 212 is located at the lower portion, and one of the inlet-outlet 110 and the opening 212 is located at the front and the other of the inlet-outlet 110 and the opening 212 is located at the back, and one of the inlet-outlet 110 and the opening 212 is located at the left and the other of the inlet-outlet 110 and the opening 212 is located at the right).

In some embodiments, the inlet-outlet 110 may be disposed at a side of the accommodation cavity, and the housing 210 may be disposed on a sidewall of the box body 100, which facilitates personnel operation. In some embodiments, the inlet-outlet 110 may be disposed at other positions, and the housing 210 may be disposed at other positions of the box body 100, such as a top of the box body 100.

In some embodiments, the transfer mechanism may include a bearing portion 310. The bearing portion 310 may be configured to bear the biological sample. The transfer mechanism may include a first transmission portion configured to drive the bearing portion 310 to move vertically and a second transmission portion configured to drive the bearing portion 310 to move horizontally. The bearing portion 310 may bear the biological sample by carrying a cryopreservation box. The transfer mechanism is simple and reliable, and has strong stability. In some embodiments, transferring may be achieved using a manipulator. That is, the transfer mechanism may be a manipulator.

In some embodiments, the first transmission portion may include a first driving device 321. An output shaft of the first driving device 321 may be connected with a first driving wheel (not shown in the figure). The first driving wheel may be connected with a first driven wheel (not shown in the figure) through a first transmission belt 323. The first transmission belt 323 may be connected with the second transmission portion through a connection member, so as to drive the second transmission portion to move vertically, which is convenient for arranging the positions of various mechanisms to adapt to a narrow space. The connection member may be connected with the box body 100 or the housing 210 through a first guide rail 326, thereby improving the stability of a movement position.

In some embodiments, the connection member may be slidably connected with the bearing portion 310. The second transmission portion may include a second driving device 331. An output shaft of the second driving device 331 may be connected with a second driving wheel 332. The second driving wheel 332 may be connected with a second driven wheel 334 through a second transmission belt 333. The second transmission belt 333 may be connected with the bearing portion 310, so as to drive the bearing portion 310 to move horizontally, which is convenient for arranging the positions of various mechanisms to adapt to the narrow space. The connection member may be connected with the bearing portion 310 through a second guide rail 335, thereby improving the stability of the movement position.

In some embodiments, the top of the housing 210 may be provided with the opening 212, which further prevents leakage of the cold energy and reduces frost inside. In some embodiments, the housing 210 may be detachably connected with the box body 100, so as to facilitate mounting, maintenance and other operations. The biological sample storage device 1000 may further include an inner liner which is disposed in the housing cavity and detachably connected with the housing 210. A heat insulation material may be disposed between the housing 210 and the inner liner, which further prevents leakage of the cold energy.

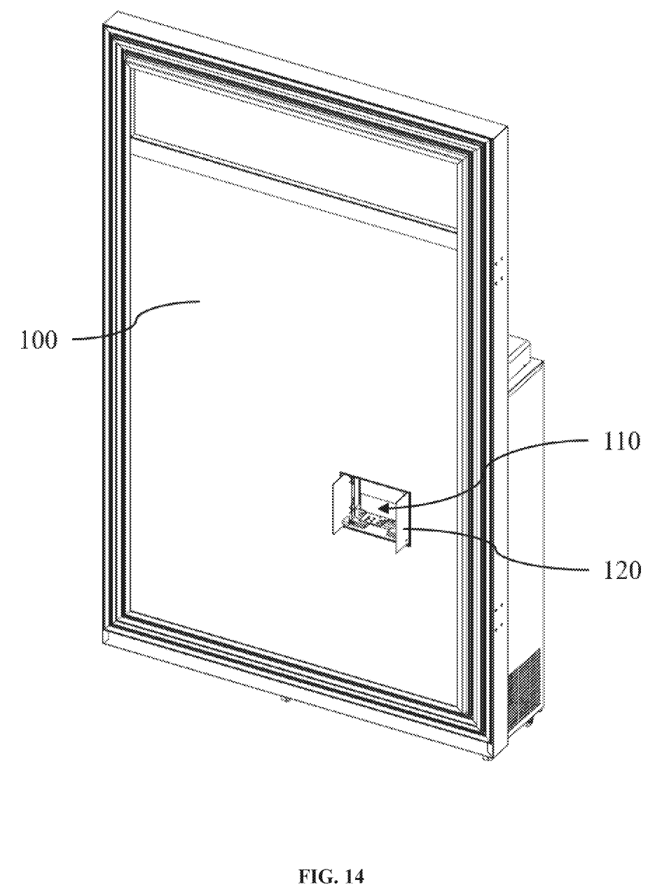

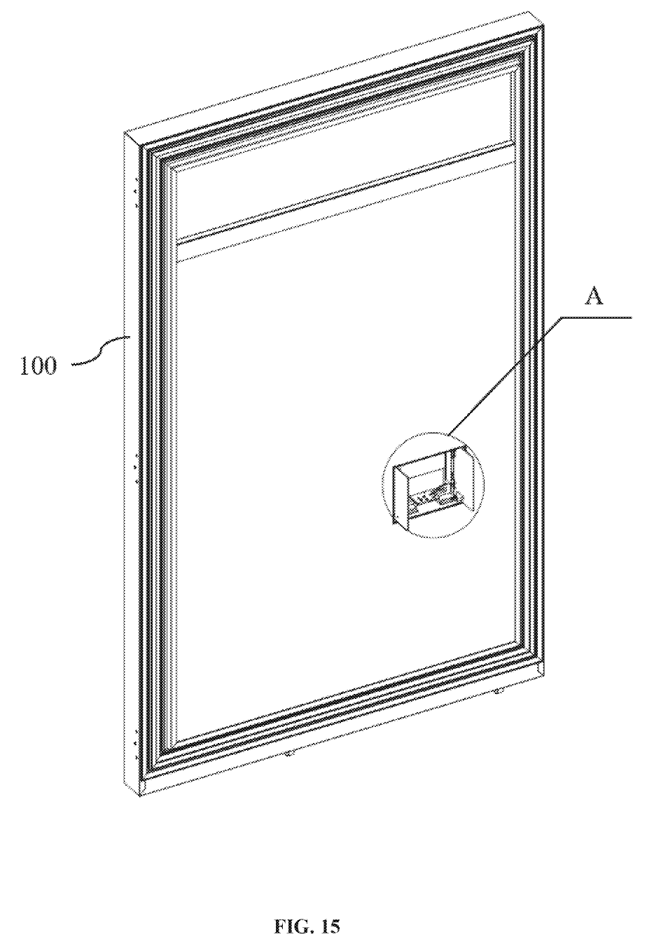

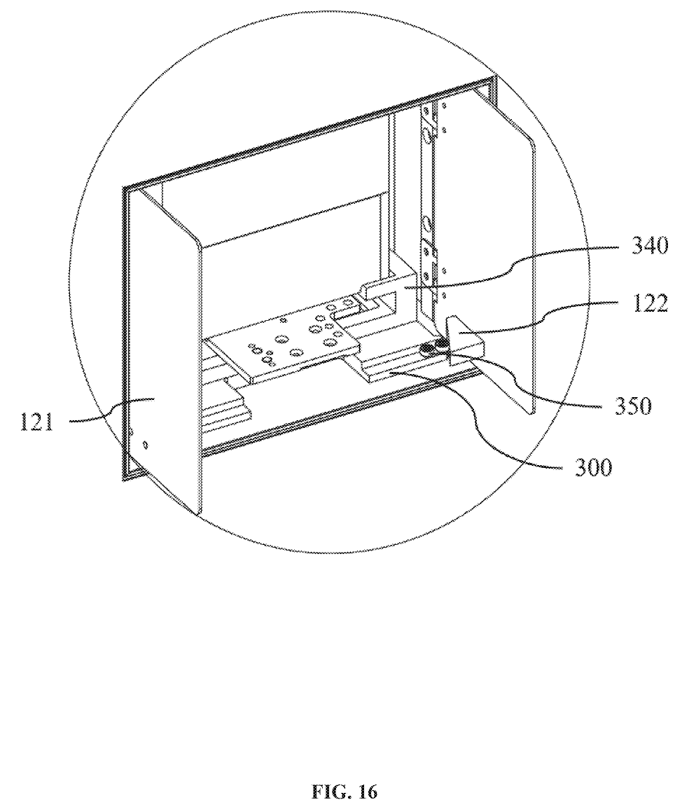

In some embodiments, the biological sample storage device is prone to leakage of the cold energy, which is not conducive to the storage of the biological sample. Some embodiments of the present disclosure provide a biological sample storage device. FIG. 14 is a schematic diagram illustrating a biological sample storage device with a portion of a box body hidden (only one panel of the box body 100 is shown) from a first viewing angle according to some embodiments of the present disclosure. FIG. 15 is a schematic diagram illustrating a biological sample storage device with a portion of a box body hidden (only one panel of the box body 100 is shown) from a second viewing angle according to some embodiments of the present disclosure. FIG. 16 is a schematic diagram illustrating an enlargement of a region A shown in FIG. 15.

In some embodiments, the biological sample storage device may include a box body, a transfer mechanism, and an inlet-outlet door body. The box body may define an accommodation cavity for storing a biological sample. The accommodation cavity may be provided with an inlet-outlet for the biological sample to enter and exit the accommodation cavity. The transfer mechanism may be disposed at the inlet-outlet of the box body. The transfer mechanism may be configured to move the biological sample into and/or out of the accommodation cavity. The transfer mechanism may be configured to have a first state of moving from outside of the accommodation cavity to inside of the accommodation cavity, and a second state of moving from the inside of the accommodation cavity to the outside of the accommodation cavity. The inlet-outlet door body may be disposed at the inlet-outlet and configured to open or close the inlet-outlet, and configured to gradually open the inlet-outlet when the transfer mechanism is in the first state and gradually close the inlet-outlet when the transfer mechanism is in the second state.

The biological sample storage device can gradually open the inlet-outlet when the biological sample is transferred to the accommodation cavity, and gradually close the inlet-outlet when the transfer mechanism withdraws from the transferring. Compared with a situation that the inlet-outlet is fully opened and then the transfer mechanism transfers the biological sample inward, and the inlet-outlet is closed after the transfer mechanism withdraws, leakage of the cold energy can be avoided, which is conducive to the storage of the biological sample.

In some embodiments, the biological sample storage device may further include an elastic member. The elastic member may be configured to connect the inlet-outlet door body and the box body, such that the inlet-outlet door body gradually closes the inlet-outlet through an elasticity of the elastic member. In some embodiments, the elastic member may be a spring, an elastic band, or other elastic members. The elastic member of the biological sample storage device provided in this embodiment can automatically close the inlet-outlet door body 120 without manual or driving mechanism, thereby improving the user experience.

In some embodiments, the inlet-outlet door body may be arranged in an inclined manner such that the inlet-outlet door body gradually closes the inlet-outlet through gravity. For example, a rotation axis of the inlet-outlet door body is at an acute angle to a horizontal plane, and an upper end of the rotation axis is closer to the outside of the accommodation than a lower end of the rotation axis. It can be understood that in order to avoid reverse rotation of the inlet-outlet door body and control an angle of the inlet-outlet door body, some limiting devices may be added. The limiting devices may be arranged on the rotation axis or on the box body, and specifically may be protrusions that interfere with the movement of the inlet-outlet door body. In some embodiments, at least one of the inlet-outlet door body or the transfer mechanism may be provided with a magnetic adsorption portion. The magnetic adsorption portion may be configured to make the inlet-outlet door body and the transfer mechanism adsorb each other such that the inlet-outlet door body gradually closes the inlet-outlet through a magnetic force. In some embodiments, the inlet-outlet door body may be driven to gradually open or close by a driving member such as a driving motor. In some embodiments, the elastic member, the inclined arrangement of the inlet-outlet door body, and the magnetic adsorption portion may be used in combination with each other.

In some embodiments, the rotation axis of the inlet-outlet door body may be inclined or horizontally arranged on a plane where a panel of the box body 100 is located, such that the inlet-outlet door body can gradually close the inlet-outlet through the gravity. For example, the rotation axis of the inlet-outlet door body may be horizontally arranged on the panel of the box body 100, and the inlet-outlet door body may naturally droop under the gravity to close the inlet-outlet.

In some embodiments, the biological sample storage device may include the transfer mechanism 300. The transfer mechanism 300 may be disposed at the inlet-outlet 110 of the box body 100, and provided with a fixing portion 340. The fixing portion 340 may be configured to fix a biological sample cryopreservation box. The biological sample cryopreservation box may be configured to store the biological sample. The transfer mechanism 300 may include a moving portion. The moving portion may be connected with the fixing portion 340 to drive the fixing portion 340 to move into or out of the accommodation cavity. The fixing portion 340 may gradually open the inlet-outlet door body 120 by pressing the inlet-outlet door body 120.

The transfer mechanism 300 improves the automation level of the biological sample storage device, and the pressing manner achieves transferring of the biological sample cryopreservation box to the accommodation cavity without fully opening the door, and the inlet-outlet door body 120 can be gradually closed during the process of moving the biological sample cryopreservation box out of the accommodation cavity, thereby avoiding leakage of the cold energy (it can be understood that the biological sample storage device requires the cold energy to store the biological sample).

In some embodiments, the moving portion may be a mechanism such as a slide rail or a slide groove. Such structures are well known to those skilled in the art, which are not repeated in this embodiment.

In some embodiments, the biological sample storage device may further include at least one roller 350 mounted on the fixing portion. Each of the at least one roller 350 may be mounted on the fixing portion 340. The fixing portion 340 may press against the inlet-outlet door body 120 through the at least one roller 350. The at least one roller 350 can reduce friction and make door opening smoother.

In some embodiments, the inlet-outlet door body 120 may include a door panel 121 and a wedge block 122. A first end of the door panel 121 may be hinged to the box body 100. A first end of the wedge block 122 may be fixed to the door panel 121. A second end of the wedge block 122 opposite to the first end of the wedge block 122 may be configured to be pressed by the at least one roller 350. The second end of the wedge block 122 may be an inclined surface. A distance between a portion of the second end of the wedge block 122 that is close to the first end of the door panel 121 and the door panel 121 may be less than a distance between a portion of the second end of the wedge block 122 that is away from the first end of the door panel 121 and the door panel 121. Therefore, a relatively small fixing portion 340 also makes the inlet-outlet door body 120 have a relatively large door opening angle.

In some embodiments, the at least one roller 350 may include a plurality of rollers 350, and arrangement directions of the plurality of rollers 350 may be different from a movement direction of the moving portion. In some embodiments, the arrangement directions of the plurality of rollers 350 may be perpendicular to the movement direction of the moving portion. In this way, different rollers 350 can press against the wedge block 122 during different movement periods of the fixing portion 340.

In some embodiments, the biological sample storage device may further include a housing component. The housing component may be connected to the box body 100 and located outside the accommodation cavity, and may cover the transfer mechanism 300 and the inlet-outlet 110. The housing component may prevent leakage of the cold energy to ensure the storage effect of the biological sample. In some embodiments, the housing component may be detachably connected to the box body 100, which facilitates mounting, maintenance, and transportation.

In some embodiments, the biological sample storage device may further include a taking and placing device disposed in the accommodation cavity and configured to place the biological sample cryopreservation box stored in the accommodation cavity on the fixing portion 340, or take the biological sample cryopreservation box fixed to the fixing portion 340 to be stored in the accommodation cavity. The taking and placing device can further improve the automation level of the biological sample storage device.

In some embodiments, the biological sample storage device may further include a second door body. The second door body may be disposed at the inlet-outlet 110 and away from the accommodation cavity relative to the inlet-outlet door body 120. The second door body can avoid leakage of the cold energy. Since the inlet-outlet door body 120 has the elastic portion, hinges, and other structures, the sealing cannot be guaranteed. Therefore, the second door body can ensure the sealing. In some embodiments, the second door body may be provided with structures such as foaming and sealing strips to avoid leakage of the cold energy. Specifically, after the second door body is opened, the fixing portion 340 is moved such that the fixing portion 340 presses against the inlet-outlet door body 120 to open the door. After the fixing portion 340 is moved out, the inlet-outlet door body 120 may be closed and then the second door body may be closed. The combination of the inlet-outlet door body 120 and the second door body can further avoid leakage of the cold energy during taking and placing.

In some embodiments, the biological sample storage device may further include a driving device connected with the second door body and configured to drive the second door body to open or close the inlet-outlet 110. Specifically, the driving device may drive the second door body to open or close in various ways such as flipping and/or translation, thereby further improving the automation level.

The beneficial effects of the embodiments of present disclosure may include but

not limited to the following content. (1) The housing cavity of the biological sample storage device and the inlet-outlet housing component thereof has the top opening. Since the cold air naturally sinks, the top opening reduces outward diffusion of the internal cold and reduces inward diffusion of the external hot air, which facilitates the storage of the biological sample. In addition, the humidity of the internal cold air is low and the humidity of the external hot air is high, and the top opening reduces the internal frost. (2) The transfer mechanism of the biological sample storage device and the inlet-outlet housing component thereof is disposed on the box body, and the inlet-outlet housing component is detachably connected to the box body. Accordingly, the transfer mechanism and other components can be inspected, repaired, replaced, etc. by disassembling the inlet-outlet housing component. The transfer mechanism and the inlet-outlet housing component are independent, i.e., the transfer mechanism is not fixed to the inlet-outlet housing component. After the transfer mechanism and other components are inspected, repaired, replaced, etc., it is easier to determine the use effect of the transfer mechanism, thereby improving the user experience. In addition, the detachable manner also facilitates the transportation of the biological sample storage device in a way that the biological sample storage device can be transported in a disassembled manner and the biological sample storage device can be assembled after being transported to the desired placed. Furthermore, the inlet-outlet housing component can also prevent the diffusion of the cold energy and ensure the activity of biological sample. (3) The positions of the inlet-outlet and the opening of the biological sample storage device are different in the at least two directions. Compared with the situation that the positions of the inlet-outlet and the opening are different in only one direction, it is difficult for the cold air to flow from the opening to the inlet-outlet and then flow out, thereby avoiding leakage of the cold energy and improving the storage effect of the biological sample. (4) The biological sample storage device can gradually open the inlet-outlet when the biological sample is transferred to the accommodation cavity, and gradually close the inlet-outlet when the transfer mechanism withdraws from the accommodation cavity. Compared with the situation that the inlet-outlet is fully opened and then the transfer mechanism transfers the biological sample inward, and the inlet-outlet is closed after the transfer mechanism withdraws, leakage of the cold energy can be avoided, which is conducive to the storage of the biological sample. (5) The refrigeration cavity of the biological sample storage device and the inlet-outlet housing component thereof is provided with the air inlet and the air outlet located below the air inlet. Due to the principle that the hot air naturally rises and the cold air naturally sinks,

the biological sample storage device and the inlet-outlet housing component thereof provided in some embodiments of the present disclosure can preferentially cool the air with a relatively high temperature, thereby improving the refrigeration efficiency, facilitating the reduction of the temperature in the housing cavity, and thus facilitating the storage of the biological sample. (6) The biological sample storage device and the inlet-outlet housing component thereof can provide the cold energy to the housing cavity, which reduces the risk of the biological sample being transferred in the housing cavity, and facilitates the preservation of the biological sample. It should be noted that different embodiments may produce different beneficial effects. In different embodiments, the beneficial effects that may be produced may be any one or a combination of the above, or any other possible beneficial effects.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by the present disclosure and are within the spirit and scope of the exemplary embodiments of the present disclosure.

What is claimed is:

1. An inlet-outlet housing component of a biological sample storage device, wherein,
the biological sample storage device includes a box body defining an accommodation cavity for accommodating a biological sample, the accommodation cavity is provided with an inlet-outlet for entry and exit of the biological sample, a transfer mechanism is disposed at a position of the box body corresponding to the inlet-outlet, and the transfer mechanism is configured to achieve transferring of the biological sample; and
the inlet-outlet housing component includes a housing defining a housing cavity, and when the housing is mounted on the box body, the housing cavity accommodates at least a portion of the transfer mechanism.

2. The inlet-outlet housing component of claim 1, wherein the housing cavity is provided with a top opening, and the transfer mechanism is configured to transfer the biological sample between the top opening and the inlet-outlet.

3. The inlet-outlet housing component of claim 2, further comprising a door body and an automatic opening mechanism, wherein:
the door body is configured to open or close the top opening; and
the automatic opening mechanism is fixed to the housing and located outside the housing cavity, wherein the automatic opening mechanism is configured to open or close the door body.

4. The inlet-outlet housing component of claim 2, further comprising:
an inner liner disposed in the housing cavity and fixedly connected with the housing, a gap being formed between the inner liner and the housing; and
a heat preservation device disposed in the gap between the housing and the inner liner and configured to reduce cold energy of the housing cavity transferred to outside of the housing cavity.

5. The inlet-outlet housing component of claim 1, wherein the housing is provided with a mounting mechanism, the mounting mechanism is configured to be detachably connected with the box body, and when the housing is mounted on the box body, the housing cavity accommodates the transfer mechanism;
the housing includes a body, and the body defines the housing cavity; and
one side of the mounting mechanism is detachably connected with the body, and the other side of the mounting mechanism is detachably connected with the box body.

6. The inlet-outlet housing component of claim 1, wherein the housing is configured to be mounted on the box body, the housing defines a housing cavity with an opening, the housing cavity is configured to accommodate the transfer mechanism, and a cold energy generation device is disposed in the housing cavity;
the inlet-outlet housing component further comprises a partition portion, wherein the partition portion is disposed in the housing cavity to separate a refrigeration cavity from the housing cavity, the cold energy generation device and a refrigeration fan are disposed in the refrigeration cavity, the refrigeration cavity is provided with an air inlet and an air outlet, and the refrigeration fan is configured to allow at least a portion of air to enter the refrigeration cavity through the air inlet and flow out of the air outlet after passing through the cold energy generation device.

7. The inlet-outlet housing component of claim 6, wherein the cold energy generation device includes an evaporator, a partition plate is disposed in the housing cavity, the partition plate is configured to separate the housing cavity into a first sub-cavity and a second sub-cavity, the partition portion is disposed in the first sub-cavity, and the second sub-cavity is provided with a compressor and a condenser which are connected with the evaporator;
the second sub-cavity is further provided with a heat dissipation fan and a water receiving box for receiving defrost water of the refrigeration cavity, and the water receiving box is located at an air inlet end of the heat dissipation fan;
the first sub-cavity is located above the second sub-cavity, and a gap is formed between a lower end of the partition portion and the partition plate to form the air outlet.

8. The inlet-outlet housing component of claim 1, wherein the housing is detachably connected with the box body.

9. The inlet-outlet housing component of claim 1, wherein the housing is configured to be mounted on the box body, the housing cavity is provided with an opening, a partition plate is disposed in the housing cavity, the partition plate separates the housing cavity into a first sub-cavity and a second sub-cavity, and the first sub- cavity is configured to accommodate the transfer mechanism;
the inlet-outlet housing component further comprises:
a refrigeration system including an evaporator, a compressor, and a condenser which are connected with each other, wherein the evaporator is disposed in the first sub-cavity, and at least one of the compressor or the condenser is disposed in the second sub-cavity; and
a water receiving box disposed in the second sub-cavity and configured to receive defrost water of the first sub-cavity.

10. A biological sample storage device, comprising a box body and an inlet-outlet housing component, wherein the box body defines an accommodation cavity for accommodating a biological sample, the accommodation cavity is provided with an inlet-outlet for entry and exit of the biological sample, a transfer mechanism is disposed at a position of the box body corresponding to the inlet-outlet, and the transfer mechanism is configured to achieve transferring of the biological sample; and
the inlet-outlet housing component includes a housing defining a housing cavity, and when the housing is mounted on the box body, the housing cavity accommodates at least a portion of the transfer mechanism.

11. The biological sample storage device of claim 10, wherein the inlet-outlet housing component includes a housing, the housing is mounted on the box body, the housing defines a housing cavity connected with the accommodation cavity through the inlet-outlet, the housing cavity is provided with an opening for the biological sample to enter and exit the housing cavity, the inlet-outlet and the opening are located at different positions in at least two directions, and a height of the opening is greater than a height of the inlet-outlet; and
the transfer mechanism is disposed in the housing cavity and configured to drive the biological sample to transfer between the inlet-outlet and the opening in the at least two directions.

12. The biological sample storage device of claim 11, wherein a side of the accommodation cavity is provided with the inlet-outlet, and the housing is mounted on a sidewall of the box body.

13. The biological sample storage device of claim 11, wherein the transfer mechanism includes a bearing portion, the bearing portion is configured to bear the biological sample, and the transfer mechanism further includes a first transmission portion configured to drive the bearing portion to move vertically and a second transmission portion configured to drive the bearing portion to move horizontally;
the first transmission portion includes a first driving device, an output shaft of the first driving device is connected with a first driving wheel, the first driving wheel is connected with a first driven wheel through a first transmission belt, and the first transmission belt is connected with the second transmission portion through a connection member to drive the second transmission portion to move vertically.

14. The biological sample storage device of claim 13, wherein the connection member is slidably connected with the bearing portion, the second transmission portion includes a second driving device, an output shaft of the second driving device is connected with a second driving wheel, the second driving wheel is connected with a second driven wheel through a second transmission belt, and the second transmission belt is connected with the bearing portion to drive the bearing portion to move horizontally.

15. The biological sample storage device of claim 10, wherein,
the transfer mechanism is configured to move the biological sample into and/or out of the accommodation cavity, and the transfer mechanism is configured to have a first state of moving from outside of the accommodation cavity to inside of the accommodation cavity, and a second state of moving from the inside of the accommodation cavity to the outside of the accommodation cavity;
the biological sample storage device further comprises a first door body, the first door body is disposed at the inlet-outlet for opening and closing the inlet-outlet and configured to gradually open the inlet-outlet when the transfer mechanism is in the first state, and gradually close the inlet-outlet when the transfer mechanism is in the second state.

16. The biological sample storage device of claim 15, further comprising: an elastic member connecting the first door body and the box body, such that the first door body gradually closes the inlet-outlet through an elasticity of the elastic member.

17. The biological sample storage device of claim 15, wherein the first door body is arranged in an inclined manner such that the first door body gradually closes the inlet-outlet through gravity.

18. The biological sample storage device of claim 15, wherein at least one of the first door body or the transfer mechanism is provided with a magnetic adsorption portion, and the magnetic adsorption portion is configured to make the first door body and the transfer mechanism adsorb to each other such that the first door body gradually closes the inlet-outlet through a magnetic force.

19. The biological sample storage device of claim 15, wherein the transfer mechanism is provided with a fixing portion, the fixing portion is configured to fix a biological sample cryopreservation box, the biological sample cryopreservation box is configured to store the biological sample, the transfer mechanism further includes a moving portion, the moving portion is connected with the fixing portion to drive the fixing portion to move into and/or out of the accommodation cavity, and the fixing portion gradually opens the inlet-outlet by pressing the first door body.

20. The biological sample storage device of claim 19, further comprising at least one roller, wherein each of the at least one roller is mounted on the fixing portion, and the fixing portion presses against the first door body through the at least one roller;
the first door body includes:
a door panel, a first end of the door panel being hinged to the box body; and
a wedge block, wherein a first end of the wedge block is fixed to the door panel, a second end of the wedge block opposite to the first end of the wedge block is configured to be pressed by the at least one roller, the second end of the wedge block is an inclined surface, and a distance between a portion of the second end of the wedge block that is close to the first end of the door panel and the door panel is less than a distance between a portion of the second end of the wedge block that is away from the first end of the door panel and the door panel;
the at least one roller includes a plurality of rollers, and arrangement directions of the plurality of rollers are different from a movement direction of the moving portion.
